(19) United States
(12) Patent Application Publication
(10) Pub. No.: US 2026/0224909 A1
(43) Pub. Date: Aug. 6, 2026

(54) MUSCULOSKELETAL PAIN RELIEVER FOR ACUTE AND CHRONIC PAIN

(71) Applicant: LITEMED INDIA PRIVATE LIMITED, Hyderabad (IN)

(72) Inventor: Surya Prakash Maguluri, Hyderabad (IN)

(73) Assignee: LITEMED INDIA PRIVATE LIMITED, Hyderabad (IN)

(21) Appl. No.: 18/869,068

(22) PCT Filed: May 24, 2023
(86) PCT No.: PCT/IN2023/050490

§ 371 Date: Nov. 25, 2024

(30) Foreign Application Priority Data
May 25, 2022 (IN) 202241030121

(51) Int. Cl. A61N 5/06

(52) U.S. Cl. CPC A61N 5/0613; A61N 2005/0627; A61N 2005/0652; A61N 2005/0659

(57) ABSTRACT

A Specially designed device which provides Pain relief therapy to the area experiencing pain. The pain relieving device is embedded with various NIR based light sources to irradiate the required pain point alleviating the pain by acting on the root cause (which is generally inside deep tissues). Also, it has a temperature control to provide safety from over-heating and an individual can perform his daily activities without any hindrance.

### DESCRIPTION OF THE INVENTION

### Technical Field of the Invention

The invention relates to pain relieving device which penetrates into the deeper layers of skin and alleviates the pain by acting on the root cause.

### BACKGROUND OF THE INVENTION

Management of acute and chronic pain is one of the biggest challenges in the healthcare industry. Better management of acute and chronic pain helps in improved quality of life in individuals. Chronic pain related diseases affect millions of people every year (at least 10% of world population). Due to improper management of pain, such conditions can impair daily routine of the individual, affecting the ability to work or perform day-to-day activities. Knee-pain is one of the most common ailments observed in elderly population due to osteoarthritis, rheumatoid arthritis, tendonitis, and other similar conditions.

In younger population, acute pain is most common due to sports injuries, muscle tears, anterior cruciate ligament (ACL) tears, extreme workouts without proper guidance etc. Overlooking of these conditions can lead to chronic pain disorders over a period of time.

The pain relieving device utilizes a pencil-like beam of electromagnetic waves of a single frequency and defined wavelength to promote tissue healing and pain relief in a broad spectrum of soft tissue injuries and diseases. The effects of the device are not thermal but photochemical reactions in cells, termed photobioactivation. This pain relieving device produces its physiological and therapeutic effects by applying enough energy to disturb local electron orbits, initiate chemical change, disrupt molecular bonds and produce free radicals at the cell membrane to control the inflammatory response, promote healing and pain relief.

The mechanism of action of Pain relieving device is not completely elucidated, but it may be related to improvements in microcirculation, inflammatory response, and adenosine triphosphate production. In addition, direct laser treatment of TrPs is believed to increase serotonin production, and trials demonstrated increased excretion of serotonin byproducts after treatment. One 4-week trial (n=60) comparing pain relieving device with dry needling and placebo laser demonstrated a significant improvement in pain at rest and with activity, as well as a rise in pain threshold in the laser-treated group. This pain relieving device has the advantage of being noninvasive and thus well tolerated, especially in patients with high tissue sensitivity. The treatment regimen and device specifications vary widely; clinicians should review the available research on the efficacy of the lasers they are considering incorporating into their practices.

Cellular chromophores responsible for the effect of visible light on mammalian cells, including cytochrome c oxidase (with absorption peaks in the near infrared) and photoactive porphyrins. Mitochondria are thought to be a likely site for the initial effects of light, leading to increased ATP production, modulation of reactive oxygen species and induction of transcription factors. These effects in turn lead to increased cell proliferation and migration (particularly by fibroblasts), modulation in levels of cytokines, growth factors and inflammatory mediators, and increased tissue oxygenation. The results of these biochemical and cellular changes in animals and patients include such benefits as increased healing in chronic wounds, improvements in sports injuries and carpal tunnel syndrome, pain reduction in arthritis and neuropathies, and amelioration of damage after heart attacks, stroke, nerve injury and retinal toxicity.

Mitochondrial cytochrome C undergoes oxidation by COX and in turn facilitates the reduction of O₂ to water as per the chemical equation below (Sarti et al., 2012).

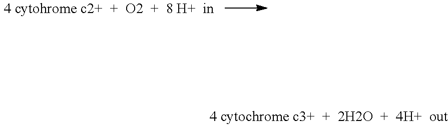

The transfer of electrons from cytochrome C to O₂ is coupled with the expulsion of 4 protons from the mitochondrial matrix across the IMM, contributing to the transmembrane electrochemical gradient that drives ATP synthesis. Nitric oxide (NO), a molecule known for its vasodilatory properties in vivo, competes with O₂ for its binding site on the Cup center of COX (Hamblin, 2018). By binding noncovalently to CUR, NO inhibits the activity of COX and hence impairs the process of oxidative phosphorylation. It is surmised that under physiological conditions, the inhibitory effect of NO may have a role in the regulation of energy production. To maximize the efficiency of resource utilization, the body reduces O₂ consumption in tissues whose metabolism is low (e.g., keratinocytes) to ensure that demand in energy-intensive tissues (e.g., neurons, hepatocytes and cardiomyocytes) can be met (Lane, 2006), hence nitric oxide is essential in improving blood circulation, which provides more oxygen and nutrients to injured tissues. Thus, infrared light hastens wound healing and stimulates the regeneration of injured tissues, reducing inflammation and pain.

One of the key health benefits of infrared therapy is improvement in cardiovascular health. Infrared light increases the production of nitric oxide, a vital signaling molecule that is important for the health of blood vessels. This molecule helps relax the arteries and prevents blood from clotting and clumping in the vessels. Aside from these, it also combats free radicals to prevent oxidative stress and regulate blood pressure.

### Use of Pain Relieving Device.

### Pain and Inflammation Management

Infrared therapy is an effective and safe remedy for pain and inflammation. It can penetrate deep through the layers of the skin, to the muscles and bones. Since infrared therapy enhances and improves circulation in the skin and other parts of the body, it can bring oxygen and nutrients to injured tissues, promoting healing. It helps case pain, relieve inflammation, and protect against oxidative stress.

Infrared light therapy can treat chronic and temporary inflammation, making it useful in the treatment of:
Osteoarthritis & Rheumatoid Arthritis
Chronic Neck Pain
Chronic Back Pain
Tendon Inflammation (Tennis Elbow, Achilles Tendon Inflammation, etc.)

Infrared light therapy is applied in the treatment of various health conditions, including trauma, joint inflammation, carpal tunnel syndrome (a common condition that causes pain, numbness, and tingling in the hand and arm and caused when the median nerve is squeezed or compressed), sciatica (the common pain that radiates along the path of the sciatic nerve, from the lower back through the hips and buttocks and down the leg), diabetic neuropathy (a type of nerve damage that can occur if you have diabetes), infrared heat lamp for muscle pain, injuries, wounds, and post-surgical incisions. Infrared therapy contributes to reduced pain, such as less muscle soreness after exercising or playing sports, as well as lightness and stiffness. The use of infrared light may help to decrease pain significantly for back pain and other common types of pain such as foot and ankle pain that restrict activity, and musculoskeletal pain in the knee joint, wrist, and other places in our bodies.

### Muscular Injuries

Infrared therapy improves the action of the mitochondria within cells, thus triggering the growth and repair of new muscles cells and tissues. In other words, infrared light can hasten the repair process after a muscle injury.

### Detoxification

Infrared therapy can be applied through saunas. Detoxifications are important since they may strengthen the immune system. At the same time, detoxification aid biochemical processes to function properly, improving food digestion. In infrared saunas, the body's core temperature increases, leading to detoxification at the cellular level.

### Potential Cancer Cure

Infrared therapy is a potentially viable cancer treatment. Studies show significant activation of nanoparticles when they are exposed to infrared radiation, rendering them highly toxic to surrounding cancer cells. One such modality is photoimmunotherapy, using a conjugated antibody-photoabsorber complex that binds to cancer cells.

Reference can be made to KR20200003526A which discloses about a heating pad made of a flexible material that relieves pain by irradiating infrared rays with a feeling of warmth by contacting a local part of the body that causes pain, and is connected to the heating pad by a cable There is one feature in a portable thermal therapy device for pain relief in a local area including a controller for controlling a heating mode of the heating pad.

Reference can be made to U.S. Pat. No. 10,071,261 which discloses about a treatment to limit the production of ROS or reactive oxygen and nitrogen species generated during, for example, reoxygenation or reperfusion of ischemic tissue. As described herein, ischemia is defined as the restriction of oxygen and nutrients to an organ or tissue. Reperfusion is defined as the process where oxygen and other nutrients are restored to ischemic tissue. The onset of reperfusion is defined as the instant in which reperfusion begins either naturally or as a result of clinical intervention. By applying the disclosed treatment at before, during, and/or after reoxygenation (e.g., prior to and during the initial minutes-hours of reperfusion) cell death and cell damage are reduced that are caused by, for instance, reperfusion injury following an ischemic event. Accordingly, an exemplary process includes applying light to an ischemic area of tissue before reperfusion and during the initial minutes-hours of reperfusion. The onset of reperfusion may be initiated by clinical intervention such as administering a clot-busting drug, inflating/deflating an angioplasty balloon, resuscitation, transfusion, or administering vaso-active drugs, among others.

Reference can be made to KR101722205B1 which discloses about a pain relief device using visible light includes a substrate on which a plurality of LEDs (Light Emitting Diodes) are dispersedly arranged, a visible light filter associated with some of the plurality of LEDs and transmitting visible light of a specific band, and the plurality of An optical filter unit in which an infrared light filter that is related to the rest of the LEDs and transmits infrared light is disposed on the same plane, a diffusion film unit which diffuses both visible light and infrared light of the transmitted specific band, and visible light of the diffused specific band and a polarizing film unit that polarizes and infrared light in a predetermined direction.

Reference can be made to US2013274834 (A1) which discloses about an invention which provides a method for treating skin tissues, the skin tissues defining an epidermal layer and a sub-epidermal layer, the epidermal layer defining a skin surface and the sub-epidermal layer extending from the epidermal layer substantially opposite to the skin surface, the method comprising: positioning a radiation source outside of the skin tissues at a predetermined distance from the skin surface; powering the radiation source so as to produce infrared radiation having a predetermined spectrum and a predetermined power; irradiating the sub-epidermal layer with the infrared radiation through the epidermal layer, the predetermined spectrum and the predetermined power being such that the infrared radiation is absorbed to a larger degree in the sub-epidermal layer than in the epidermal layer.

Reference can be made to EP2776128 (B1) which discloses about a light therapy device as defined in the claims. It may be substantially the same as that described in United States Patent Application Publication No. 2011/0144724 (Benedict) but which is capable of being formed into and retaining a desired shape (e.g., a curved or twisted shape) without the need for strap(s) or other restraining apparatus to hold the device in the desired shape. For at least some applications, the improved formability of the devices of the present invention enables such devices to be pre-formed to various shapes to ensure that many or all of the light emitters on the device will be within a therapeutically optimal distance (in some cases—as close as possible without touching) from the affected body surface. By positioning the light emitters within the therapeutically optimal distance from the body surface, factors such as the depth to which therapeutic wavelength(s) of light penetrate the subject's body

### BRIEF DESCRIPTION OF DRAWINGS

FIG. 1 Placement of Symmetric—Equilateral triangle pattern for LED arrangements (Black points are NIR LEDs and Red points are RED LEDs)

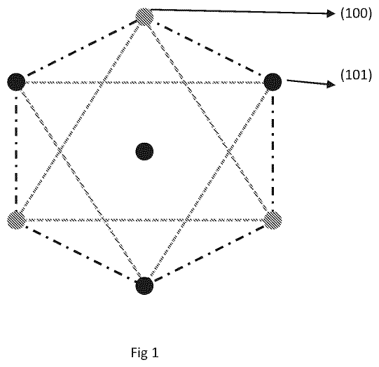

FIG. 2 Plate with apertures for LEDs in pattern

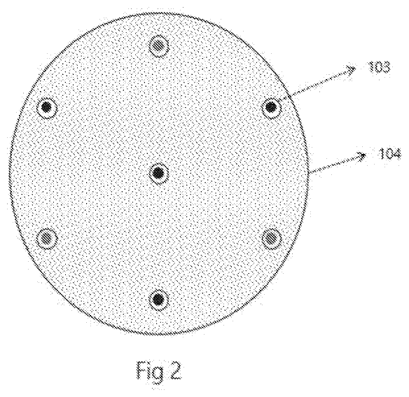

FIG. 3 Superimposition of light rays with equilateral triangle patterns

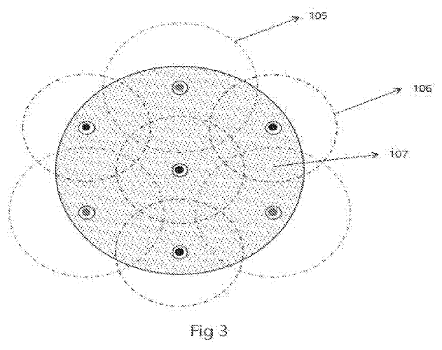

FIG. 4 Light rays getting reflected due to the plate back to the human skin.

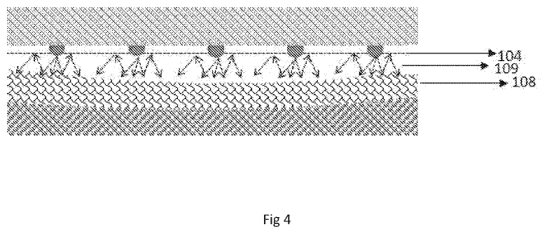

FIG. 5 Irradiation of device on Back region showing active irradiation area of the device.

FIG. 6 Irradiation of device on Shoulder region showing active irradiation area of device.

FIG. 7 Irradiation of device on Thigh region showing active irradiation area of device.

FIG. 8 Transmission of device irradiance at red and near-infrared region through the human wrist.

FIG. 9 Transmission of device irradiance at red and near-infrared region through the human palm.

FIG. 10 Transmission of device irradiance at red and near-infrared region through the human knee.

FIG. 11 Transmission of device irradiance at red and near-infrared region through the human finger.

FIG. 12 Transmission of device irradiance at red and near-infrared region through the human abdominal region.

### PROBLEM TO BE SOLVED

Current non-pharmacological methods orthopaedic bands, heating pads and other non-invasive approaches.

Orthopaedic bands provide only support only to the joints of areas experiencing pain. This will not alleviate the pain by providing action on the root cause. Hence, only a momentary (for a short time) control over pain can be achieved. These bands can cause discomfort when worn for a longer period of time.

Heating pads cannot be applied to the areas with swelling, as they tend to increase the pain. If heating pads are used for longer durations than the stipulated time period, there is a strong chance of overheating and damage to the skin.

Devices with current technological advancements do not provide any support to the areas experiencing pain, also a few of these devices require trained personnel or healthcare professional to provide the therapy. Moreover, these devices need multiple session, each consuming a huge amount of time during the therapy. Furthermore, they are highly uneconomical and are available mostly in healthcare centres. Also, these devices either do not expose the exact pain point or require a hand-held applicator to expose the exact pain point, resulting in poor adherence.

### Object of Invention

Main object of the invention is to provide Near IR device which can penetrates into the deeper layers of skin and alleviates the pain.

Another object of the present invention is to provide Near IR device which docs not damage skin.

Another object of the present invention is to provide optimum configuration or pattern of LED's for superimposing light rays from LED's to deliver maximum possible dose to the deepest of skin layers.

### General Statement of Invention

Pain relieving device is used to induce pain-relieving action which can be achieved by initiation of repair at intra cellular level where various pain pathways are activated during the treatment. This provides long-term pain-relieving action unlike pharmacological therapy.

### SUMMARY OF THE INVENTION

The device of the current invention provides pain relief therapy to the area experiencing pain. The pain relieving device is embedded with various NIR based light sources to irradiate the required pain point alleviating the pain by acting on the root cause (which is generally inside deep tissues). Also, it has a temperature control to provide safety from over-heating and an individual can perform his daily activities without any hindrance.

### DETAILED DESCRIPTION OF THE INVENTION

This pain relieving device is based on the bi-phasic dose response curve, which infers that if higher dose is given it can lead to detrimental effect, and if low dose is given the sub-therapeutic effect is seen. Hence, there is a need for optimum dose to be delivered to achieve the maximum therapeutic effect. Appropriate LED arrangement pattern is one of the most critical aspect of delivering optimal dose to various body parts.

In one embodiment the device is designed in a unique LED array pattern, where the LEDs are placed in two symmetrical-equilateral triangle pattern, where both the equilateral triangles have overlapped each other in such a way to form a common centroid (where another LED is placed at this position), and other LEDs equidistant from each other as well as equidistant from the centroid. First equilateral triangle (105) has LEDs with an exposure angle of 90-150 degrees, while the second equilateral triangle (106) and centroid has LEDs with exposure angle of 60-120 degrees. This arrangement helps in super imposition of light rays from one LED with the light rays from its adjacent LEDs creating multiple illuminated areas with superimposed light rays (107) acting as a light source on its own to deliver the dose to maximum possible exposure area and to the deepest of skin layers (up to Hypodermis and Sub-cutaneous tissue) (108), the device also is capable to emit radio wave of a required frequency along with Red (100) and NIR (101) light rays to reach deeper tissues after the sub-cutaneous layer to attain the pain relief action at much deeper levels

In one embodiment a plate (104) of high reflectivity is placed after the LEDs with perforations for the light rays to pass through without any obstruction. The plate (104) is highly reflective on the outside (the side facing the skin). Once the light rays interact with human tissue, there will be absorption, refraction, and reflection. These reflected rays (109) are diverted back to the human tissue to provide maximum utilization of the light rays for effective pain relief action in shorter time period. With the symmetrical LED pattern and plate arrangement, superimposition of the light rays (107) is possible making sure that the complete human skin area (108) covered by the device is exposed with light rays emitted by the LEDs, while the stray light is collected and directed back towards the human skin (108) ensuring maximum exposure area coverage and maximum utilization of the light rays to provide highest dose delivery to the human skin (108) in order for the light to reach the deepest of skin (108) layers (up to Hypodermis and Sub-cutaneous tissue) maximum light dose can be delivered in coupling of the optical LED symmetrical pattern with the plate (104) for stray light reflection.

In another embodiment a highly transmissive layer is placed after the plate (104) which also prevents excessive heat conduction to human tissue. Also, over time this transmissive layer can go through wear and tear due to rough use of device or placement on any highly rough and irregular surfaces, as the wear and tear will affect the light transmission efficiency of the transmissive surface. To avoid this the perimeter of the device is extended downwards a little with highly reflective surface on the inside. This will prevent the wear and tear of the transmissive layer and further reflect back the light escaping which is reflected back by human tissue outside the skin (108). This will provide increased shelf life of the transmissive layer and maximum light utilization for pain relief.

In another embodiment the pain relieving device for pain relief is a result of intracellular mechanisms which can be affected due to temperature changes. Prolonged exposure of skin (108) to unusual temperatures, results in change in physiological skin (108) parameters and homeostasis of the body. Hence, there is a need to address excessive heating of skin (108) due to Red (100) and NIR (101) light exposure. This can be accomplished by integrating thermo regulation sensors to detect the temperature of skin (108). This thermoregulation of skin (108) is very important in prevention of burns and other physical damage to the skin (108). To achieve the goals of treatment with the pain-relieving device, temperature change is compensated by a specialized heat dissipation mechanism inside the device based to prevent the increase in temperature at the Red (100) and NIR (101) light exposed area. This will enable us to provide continuous therapeutic action over the exposed area by preventing the heat accumulation and optimum dose delivery throughout the applied area.

In another embodiment the device has multiple dose delivery modes for different pain conditions, or different dose delivery modes according to the placement of device on various body parts such as knee, lower back, stomach, shoulder, elbow, wrist etc. These dose delivery modes can be controlled by using the smart phone app provided for the device. Once the device is placed on the respective body part, the dose required for pain relief at that particular point can be controlled using the app. The device illumination time also can be controlled by the app.

The wavelengths used are chosen from the biological band and have specific viewing angles in a range of 60°-120°. These are arranged in a unique array pattern resulting in constructive superimposition. Each individual LED of area 0.2 cm² synergizes the effect of each other due to constructive superimposition thereby enabling device to act as a unified irradiation source with an active irradiating surface area of 15.19 cm², ultimately resulting in a penetration depth of 70 mm.

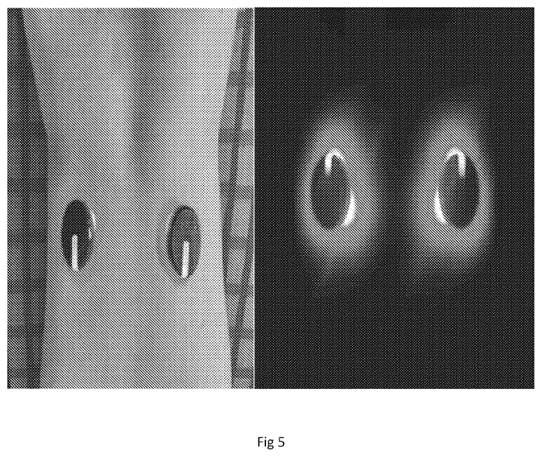

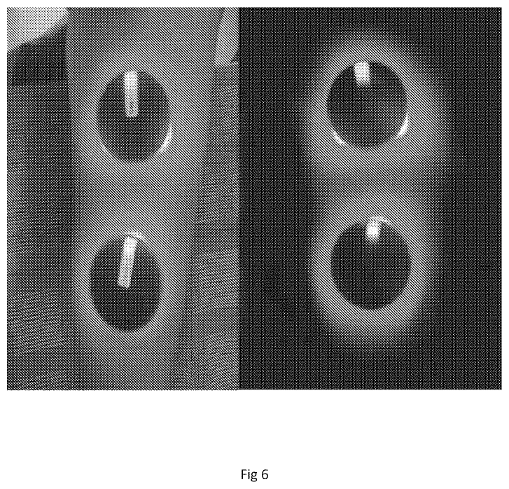

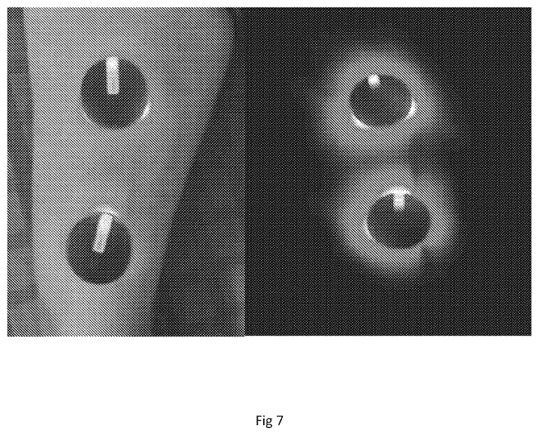

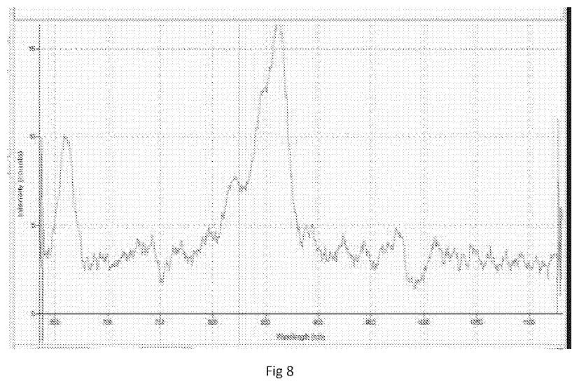

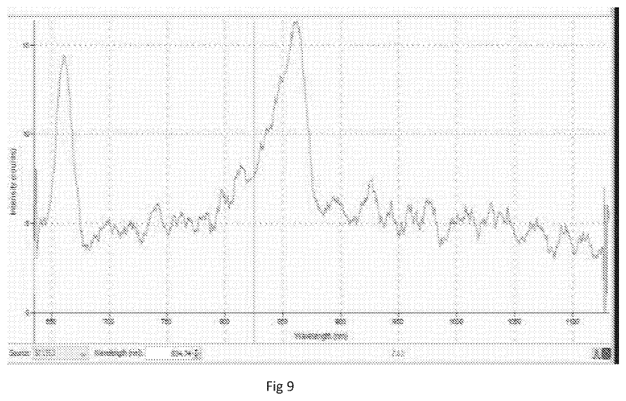

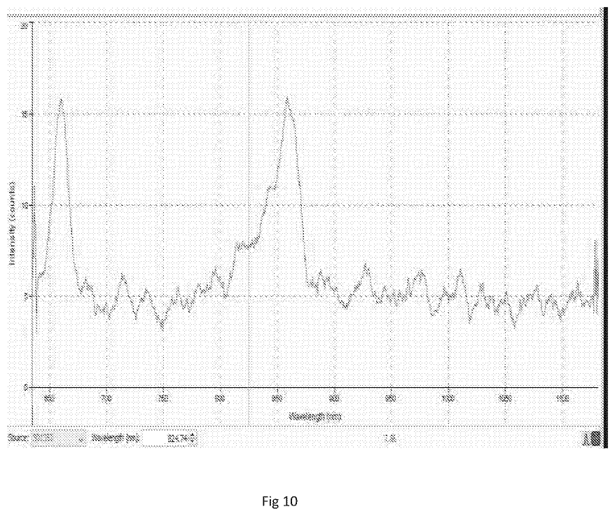

Optical simulations were carried out in order to design the most efficient optical arrangement of the Red and NIR sources for attaining a penetration depth of 70 mm in the human body. The simulations were performed by modelling various human skin tissues embedded in with their optical properties (such as Epidermis, dermis, hypodermis, subcutaneous tissue or fat layer, and muscle tissue with varying thickness and optical properties). These tissue models were subsequently irradiated using optical source models of Red and Near-infrared ranges, as well as detectors were placed in different patterns to study the optical interactions with human tissue layers. By performing these simulations device LED array placement has been designed to produce constructive superimposition to penetrate a depth of 70 mm in the human body as shown from FIG. 5 to FIG. 10.

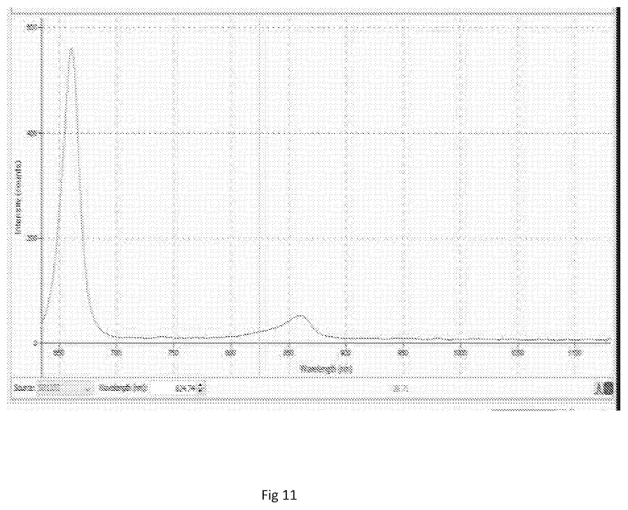

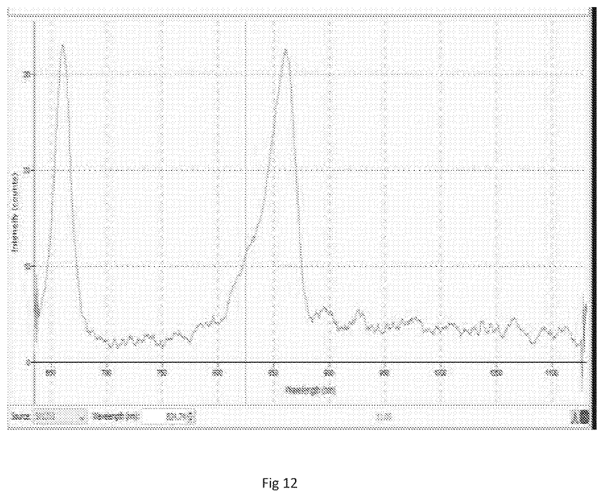

Spectrophotometric tests were conducted on different parts of the human body using Ocean optics spectrophotometer-STS NIR. This was done to evaluate the penetration depths of this device wavelengths at various body parts using the array system designed through simulations. Body parts exposed with this instant device irradiation were human wrist, finger, palm, knee, and abdominal region. Each of the body region was exposed using instant device and an optical probe of the spectrophotometer was placed on the opposite side of the body region to detect the light intensity passing through the body part. The detection of the irradiance at the end of body part by the spectrophotometer indicates that the wavelengths emitted by instant device LED array could penetrate a depth of 70 mm, allowing it to provide pain relief for deep-seated chronic pains as depicted from FIG. 11 to FIG. 15.

Table 1 consists of pain relief data for subjects with various pain conditions of lower back. Each subject is diagnosed with deep seated pain conditions such as L4-L5 Prolapsed Intervertebral Disk (PIVD), L4-L5 Facet Arthropathy, L4-L5 Annual tear, and Paraspinal muscle spasm. These deep-seated pain conditions have the origin of pain with a depth of more than 20-25 mm from the skin, which is much deeper than the subcutaneous tissue layer. Subjects are randomly distributed into 2 groups of control and treatment, where the treatment group is provided with actual device and control are provided with placebo. Intensity of pain is recorded using Visual Analog Scale (VAS-Score is a scale on the rating of Zero to Ten, where “Zero” is No pain and “Worst Possible Pain” is Unbearable Pain) before and after the treatment session of the subject. Based on the VAS Score the percentage pain relief for the subjects is derived using a statistical formula. An average pain relief of 70% was observed in the treatment group and 6.86% in control group, with such deep-seated pain conditions of more than 20-25 mm. While current devices with such NIR irradiation have a penetration of up to 5 mm or less, pain relief of about 70% was observed at a depth of more than 20-25 mm. This device can provide a penetration depth of up to 20-70 mm from the skin level.

TABLE 1

| | | | | | |
|---|---|---|---|---|---|
| | | | VAS | VAS | |
| | | | Score | Score | |
| | | | Before | After | Percentage |
| | | Pain | Treat- | Treat- | Pain |
| Cause of pain | Condition | Site | ment | ment | Reduction |

TREATMENT DATA FOR LOWER BACK PAIN

| | | | | | |
|---|---|---|---|---|---|
| L4-L5 PIVD | Acute | Lower Back | 5 | 3 | 40 |
| L4-L5 facetal | Chronic | Lower Back | 4 | 0 | 100 |
| arthopathy | | | | | |
| L4-L5 PIVD | Chronic | Lower back | 5 | 2 | 60 |
| L4-L5 Annular | Chronic | Lower Back | 5 | 1 | 80 |
| Tear | | | | | |

| | |
|---|---|
| FINAL PAIN RELIEF | 70% |

CONTROL DATA FOR LOWER BACK PAIN

| | | | | | |
|---|---|---|---|---|---|
| facetal | Chronic | Lower back | 7 | 7 | 0 |
| arthopathy | | | | | |
| Para spinal | Acute | Lower Back | 5 | 4 | 20 |
| muscle spasm | | | | | |
| L3-L4 PIVD | Chronic | Lower back | 3 | 3 | 0 |
| L4-L5 facetal | Acute | Lower Back | 5 | 5 | 0 |
| arthopathy | | | | | |
| L4-L5 facetal | Chronic | Lower Back | 7 | 6 | 14 |
| arthopathy | | | | | |

| | |
|---|---|
| FINAL PAIN RELIEF | 6.86% |

We claim:

1. A Musculoskeletal pain reliever for acute and chronic pain, comprising of an array of LED source, a plate embedded in a pod like structure, wherein the said array of LED's results in more than one symmetrical pattern for superimposition of the light rays emitted from the array of LED's capable of penetrating hominid tissue.

2. A Musculoskeletal pain reliever for acute and chronic pain as claimed in claim 1, wherein the said LED source is selected from the group comprising of NIR and Red LED's.

3. A Musculoskeletal pain reliever for acute and chronic pain as claimed in claim 1, wherein the said plate is placed anterior to the LED's and the said plate is perforated for the LED's placed posterior to the plate.

4. A Musculoskeletal pain reliever for acute and chronic pain as claimed in claim 3, wherein a highly transmissive layer is placed at the exterior of the perforated plate for preventing excessive heat conduction from the device.

5. A Musculoskeletal pain reliever for acute and chronic pain as claimed in claim 1, wherein the LEDs are placed in two symmetrical equilateral triangle patterns, wherein both the equilateral triangles have overlapped each other in such a way to form a common centroid.

6. A Musculoskeletal pain reliever for acute and chronic pain as claimed in claim 5, wherein the first equilateral triangle (105) has LEDs with an exposure angle of 90-150 degrees.

7. A Musculoskeletal pain reliever for acute and chronic pain as claimed in claim 5, wherein the second equilateral triangle (106) and centroid has LEDs with exposure angle of 60-120 degrees.

8. A Musculoskeletal pain reliever for acute and chronic pain as claimed in claim 1, wherein the said arrangement of LED's for a symmetrical pattern for superimposition creating multiple illuminated areas with superimposed light rays.

9. A Musculoskeletal pain reliever for acute and chronic pain as claimed in claim 1, wherein the said light rays emitted from the array of LED's penetrates hominid tissue to a depth ranging from 20 mm to 70 mm.

10. A Musculoskeletal pain reliever for acute and chronic pain as claimed in claim 1, further comprises of dose delivery modes controlled by a smart phone app provided for the device wherein, once the device is placed on the respective body part, the dose required along with the duration of exposure for pain relief at that particular point is controlled using the app.
